(19) United States
(12) Patent Application Publication
(10) Pub. No.: US 2018/0025480 A1
(43) Pub. Date: Jan. 25, 2018

(54) APPARATUS AND METHOD FOR ANALYSIS OF GROWING ITEMS

(71) Applicant: The Technology Research Centre Ltd., Newark (GB)

(72) Inventors: Laurence Dingle, Lincolnshire (GB); Chris Roberts, Leicestershire (GB); Michael Kalyn, Lincolnshire (GB); David Samworth, Lincolnshire (GB)

(73) Assignee: The Technology Research Centre Ltd., Newark (GB)

(21) Appl. No.: 15/549,046

(22) PCT Filed: Feb. 5, 2016
(86) PCT No.: PCT/GB2016/050281

§ 371 Date: Aug. 4, 2017

(30) Foreign Application Priority Data
Feb. 5, 2015 (GB) 1501882.3

(51) Int. Cl. G06T 7/00; G06T 7/73; G06T 7/10; G06F 17/30; G06K 9/20; G06T 7/80; A01G 1/00; G01N 21/84; G06K 9/62

(52) U.S. Cl. CPC G06T 7/001; G01N 21/84; G06T 7/74; G06T 7/10; G06K 9/623; G06K 9/209; G06T 7/85; A01G 1/00; A01G 1/001; G06F 17/3028; G01N 33/025

(57) ABSTRACT

A vehicle-mounted apparatus for analysis of growing items having a camera configured to capture multiple sequential images of growing items, a memory comprising an image buffer for storing the images, a processor, software, which when executed on the processor is configured to repeatedly call an image from the image buffer, process the image to identify individual growing items to create item data relating to individual growing items, and update a growing item database by adding the item data wherein the software is configured to run whilst the camera captures further images, and the images are discarded once processed.

### CROSS-REFERENCE TO RELATED APPLICATIONS

This application claims priority to PCT/GB2016/050281 filed Feb. 5, 2016, and GB1501882.1 filed Feb. 5, 2015, each of which is incorporated herein by reference in its entirety.

### FIELD

The present invention is concerned with an apparatus and a corresponding method for the analysis of growing items. More particularly, the present invention is concerned with an apparatus and method for counting and measuring various qualitative characteristics (e.g. size, colour, appearance) of edible items during growth (i.e. in situ on the plant).

### BACKGROUND

The ability to count growing items such as vegetables and fruit (including the fruiting bodies of fungi) before they are harvested is highly desirable. If the farmer can accurately predict the yield, then he or she can use this to accurately secure the necessary purchase orders from his or her customers. Accurate prediction of yield mitigates the risks associated with over-prediction (resulting in a shortfall) or under-prediction (resulting in wastage). The earlier the yield can be measured, the better the farmer can plan for the upcoming harvest.

One example of this is in “top fruit” such as apples, pears and kiwis. In apple production for example, in addition to absolute quantities of fruit, there is a high demand for “class 1” fruit—i.e. fruit which display certain desirable characteristics to the consumer (in terms of size, colour etc.). A single orchard can have outputs from a single cultivar which vary by up to 60% year on year, both on the overall tonnage yield per hectare, and on the percentage of substandard fruit. Rejected fruit (i.e. not meeting the “class 1” criteria) goes to waste or low value processing and typically loses up to 80% of its value.

The market price of apples is dependent on crop quality and the crop yield declared by the growers and wholesalers. These estimates tend to be inaccurate with a variation of typically ±20% of final yield. This has a major impact on market price. Quotas are agreed with the supermarkets early in the season (whilst the fruit is still growing) and must be fulfilled. Over-estimating the yield may mean purchasing imports (at late-season high prices) to cover the shortfall, while under-estimating yield, means losing profits by selling excess crop to low value outlets or for low value uses such as animal feed.

As well as prediction of crop yield, a key part of orchard management practice is to know when and how to ‘thin’ crops to promote selective growth. After they flower, apple trees undergo the “June drop”, during which apparently healthy (but immature) fruit are shed by the tree. This is thought to be a mechanism by which the tree matches the number of fruit to its natural resources to provide a healthy mature yield. After the drop, it is understood that the remaining number of fruit determine both the apple yield for that season, as well as influence the yield for the following season. Therefore the farmer may wish to remove further fruit at this stage to ensure the optimum number is left. As such, counting immature fruit after the June drop is beneficial for orchard management.

There are several challenges associated with analysis of all types of fruit and vegetables, and in particular top fruit (by which we mean fruit grown on trees, such as apples).

As well as counting fruit, it is very useful for certain characteristics to be identified. For example size, colour, blemishes etc. may be of interest to the farmer to understand the qualitative characteristics of his crop. Although a fruit may match the size criteria for “class 1”, a blemished or diseased fruit is of little value.

Another challenge is that the fruit on trees and bushes may be obscured by leaves, making counting and analysis by a vision based system difficult. This problem can be mitigated by taking several overlapping images of the location from multiple positions. It is conceivable that a leaf which obscures an apple in one position of the camera, would not in a further position due to parallax. Even if this technique is adopted, there are inherent problems—e.g. how to avoid counting apples which appear in both images twice, and how to handle the amount of image data created by the increased number of images required by this technique. Mobile, vehicle mounted imaging systems need to be rugged to withstand vibration. This limitation usually results in limited storage—e.g. by using solid state drives. The amount of image data required to accurately map an orchard of, say, 70 hectares, is far in excess of the solid state drive available in commercial ruggedized PCs. Image resolution could be compromised to reduce file size, but this would lead to a reduction in accuracy. Therefore it is desirable to be able to use high frequency, high resolution imaging to ensure accurate results.

Also, false identification of fruit is problematic. In orchards, pollinator trees may be planted to aid fruit production. These pollinator trees are usually apple trees of a different species, and may bear fruit which is of no commercial value. The pollinator tree fruit needs to be identified and ignored.

The speed at which fruit is counted is also important. If a counting apparatus was to travel at 5 kmph it would typically take 2-3 days to cover a 40 hectare orchard. Therefore any increase in speed is beneficial.

Prior art vision systems have been proposed. US2013/0204437 discloses an agricultural system for harvesting utilising a scout robot followed by a harvesting robot. The scout robot uses cameras and image analysis to identify fruit for harvesting. The system is only used for harvest though, and is not suitable for yield prediction or orchard management because only mature fruit are identified. The proposed system is also provided on a bespoke vehicle, which would be costly to manufacture.

What is required is an apparatus and method of analysing growing items which provides an accurate representation of the quantitative and preferably qualitative characteristics of growing agricultural items.

### SUMMARY OF THE INVENTION

According to a first aspect of the invention there is provided an apparatus for analysis of growing items, the apparatus suitable for installation on a vehicle and comprising:

at least one camera configured to capture multiple sequential images of growing items;

a memory comprising an image buffer for storing the images;

a processor;

software, which when executed on the processor is configured to repeatedly:
call an image from the image buffer;
process the image to identify individual growing items to create item data relating to individual growing items;
update a growing item database by adding the item data;

wherein the software is configured to run whilst the camera captures further images, and at least some of the images are discarded once processed.

According to a second aspect of the invention there is provided a method of analysis of growing items comprising the steps of:

providing a vehicle;

providing an apparatus mounted on the vehicle, the apparatus comprising:
at least one camera configured to capture multiple sequential images of growing items;
a memory comprising an image buffer for storing the images;
a processor;
software, which when executed on the processor is configured to repeatedly:
   call an image from the image buffer;
   process the image to identify individual growing items to create item data relating to individual growing items; and
   update a growing item database by adding the item data;

causing the vehicle to move, and whilst the vehicle is in motion;
running the software whilst the at least one camera captures images; and
discarding at least some of the images once processed by the software.

Advantageously, this apparatus and method allows real-time processing of the images which are being captured by the camera. Once analysed, the images can be discarded and/or overwritten which means that significant on-vehicle storage is not required. The invention therefore enables the use of low-cost commercially available ruggedised hardware utilising e.g. solid state drives. The invention also enables a high frame rate of high resolution images to be processed, which leads to more accurate results. The use of ruggedised, commercially available hardware means that the system can be made compact, which means it can be attached to a vehicle which is coincidentally carrying out another operation in the orchard—e.g. spraying.

By “discarded” we mean that the images are deleted and/or overwritten. In other words, the vast majority of images are not retained in the apparatus (save for those images which may be retained in the buffer, or retained for e.g. validation or manual audit purposes). It is envisaged that at least 95% of the images are discarded. According to the invention the available memory is significantly less than the amount of image data which is captured throughout the operation, but is sufficient to store the item data generated (which is, by definition, smaller in size). Therefore by filtering the image data to produce item data in real time, the amount of storage required is reduced, and rugged, cheaper, commercially available solid state drives can be used.

Preferably the system comprises a GPS receiver for generating location data, in which the software is configured to combine the item data with location data and update the growing item database with the combined item data and location data. This provides the farmer with a “map” of crop distribution. As well as GPS, absolute other known locators can be implemented. Preferably the system is accurate to at least 2 m, more preferably to less than 1 m.

Preferably the software is configured to identify and disregard growing items which were previously added to the growing item database. This enables the system to take multiple images of the same location (which is beneficial for identifying obscured items), but ensures that they are not counted twice as a result.

Preferably the software is configured to carry out the following steps:

to identify a growing item in a first image;

to predict the position of the growing item in a second, subsequent image; and,

to disregard the growing item in the subsequent image if the position matches the predicted position within a predetermined margin of error.

Preferably the step of predicting the position of the growing item is carried out using a velocity vector based on the speed and direction of a vehicle on which the apparatus is mounted. This may be provided by the vehicle speedometer, or by a GPS system. More preferably the velocity vector is refined by calculating the speed and direction of the growing object based on changes in its position between at least two images. The calculated speed may be checked against the GPS speed of the vehicle from time to time to ensure accuracy.

This allows accurate avoidance of e.g. duplicate counting.

Preferably, images are removed from the buffer when they are called by the software. The image buffer may be a ring buffer.

The buffer may be expandable, but may have a maximum size, upon reaching which a user is alerted.

Preferably the apparatus comprises a display, and the apparatus is configured to display the growing item database on the display as it is updated. More preferably the apparatus is configured to show a visual representation of the growing item database as it is updated. This provides the driver of the vehicle with real time information. Preferably the visual representation is a map of growing items.

In terms of image processing, the software is preferably configured to identify seed pixels in the image using a co-occurrence matrix. Such matrices are well suited to identifying areas of images with specific qualities, which can identify the presence of e.g. fruit. Preferably seed pixels are identified from the co-occurrence matrix based on a set of seed parameters determined by a user-selection option, which may include a type (e.g. apples) and species (e.g. braeburn) of growing item. In this way, irrelevant fruit (such as that of pollinator trees) can be ignored.

Preferably the software is configured to carry out a segmentation step to identify further candidate pixels proximate the seed pixels. A combined approach using both a flood fill and a spiral algorithm based on a set of segmentation parameters less stringent than the seed parameters is used. This provides a region of pixels representing the item, which can be used to filter and determine certain qualities of the item for further analysis. Advantageously, the combined approach using two algorithms provides improved growing item identification.

Preferably the software is configured to filter individual regions of pixels based on size, and regions below a certain size are disregarded. Preferably the software is configured to produce item data by analysis of at least one of the size, shape, colour and aspect ratio of the regions of pixels. Preferably the software is configured to identify partially obscured growing items and to extrapolate at least one of their size and shape.

Preferably a further processor (specifically a GPU) is provided which carries out one or more of the steps of image processing. This split processing allows much faster analysis and allows the vehicle to travel at higher speeds.

Preferably the steps of image processing are carried out using a set of parameters determined by a user input. Preferably the user input contains information identifying the type and/or breed of growing item. The apparatus preferable comprises a user input device for providing the user input.

Two cameras having an overlapping field of vision may be provided, in which the software is configured to calculate a depth of field between the cameras and the growing items using a stereoscopic measuring technique based on an overlap between simultaneously captured images. The software may be configured to process the image to size individual growing items based on the calculated depth of field, and in particular individual growing items outside the overlapping field of vision may be sized based on the calculated depth of field. This allows for speedy processing of the images without having to calculate the depth of field for each item. It also avoids the need for stereoscopic vision across the whole scanned area.

Preferably the software is configured to determine a status of the buffer and output a speed feedback signal based on the status of the buffer. This may be via a HMI or direct to a speed control of the vehicle.

Preferably the software is configured to count the number of growing items on a given plant, plurality of plants or part of a plant; compare the counted items to an expected count; and then if the counted number of items differs from the expected count by a first predetermined margin, prompt a user to perform a calibration process. This may involve a manual count to verify and/or modify an obscuration factor. Preferably the expected count is a cumulative mean of previous counts with the predetermined margin being a factor of the standard deviation of those previous counts.

The invention also provides a method to improve the planning for harvesting growing items comprising the steps of:

analysing an area of growing items using a method according to the second aspect; and

harvesting the growing items.

Preferably the method predicts an optimum harvest time and the items are harvested at the optimum harvest time.

According to a third aspect of the invention there is provided a method of analysing growing item image data comprising the steps of:

providing two growing item images from spaced apart viewpoints, the images being partially overlapped to define an overlap;

determining the depth of field of vision of a growing item within the overlap region based on a stereoscopic technique;

determining the size of the growing items within at least one of the growing item images, outside of the overlap, using the determined depth of field of vision.

Preferably all of the growing items in the growing item images are sized using the determined depth of field of vision.

This allows for significant economy in processing time compared to determining the depth of each item. It also avoids the need for stereoscopic imaging across the full field of vision.

Preferably the method includes the steps of:

providing at least one further growing item image taken at a different time to the growing item images;

determining the size of the growing items within at least one further growing item image using the determined depth of field of vision.

According to a fourth aspect of the invention there is provided a vehicle having an apparatus for analysis of growing items, the apparatus comprising:

a camera configured to capture multiple sequential images of growing items;

a memory comprising an image buffer for storing the images;

a processor;

software, which when executed on the processor is configured to repeatedly:

call an image from the image buffer;

process the image to identify individual growing items to create item data relating to individual growing items;

wherein the software is further configured to:

determine a status of the buffer;

output a speed feedback signal based on the status of the buffer.

According to a fifth aspect there is provided a method of controlling the speed of a vehicle, comprising the steps of:

providing a vehicle having an apparatus for analysis of growing items, the apparatus comprising:
a camera configured to capture multiple sequential images of growing items;
a memory comprising an image buffer for storing the images; and,
a processor;

using the processor to process an image from the buffer to identify individual growing items and to create item data relating to individual growing items;

determining a status of the buffer;

outputting a speed feedback signal to the vehicle based on the status of the buffer.

This allows the vehicle to be run at the optimum speed, and recognises that the processor load is dependent on the number of growing items in the field of vision. Therefore the invention allows faster speeds in sparse areas, and requests slower speeds in dense areas to ensure that the processor is used efficiently, and no loss of data is experienced by e.g. unprocessed images being overwritten.

Preferably in determining the status of the buffer the apparatus determines the amount of data in the buffer. Preferably the vehicle has a user display, in which the feedback signal is fed to the user display.

If the amount of data in the buffer is too large, the user display provides the user with information relating to the speed of the vehicle being too fast. Similarly if the amount of data in the buffer is too small, the user display provides the user with information relating to the speed of the vehicle being too slow.

The feedback signal may be fed to a vehicle speed controller to automatically control the speed of the vehicle.

According to a sixth aspect of the invention there is provided a vehicle having an apparatus for counting growing items, the apparatus comprising:

a camera configured to capture multiple sequential images of growing items;

a processor;

software, which when executed on the processor is configured to repeatedly:

process the image to count the number of growing items on a given plant, plurality of plants or part of a plant;

wherein the software is further configured to:

compare the counted items to an expected count; and,

if the counted number of items differs from the expected count by a first predetermined margin, prompt a user to perform a calibration process.

According to a seventh aspect of the invention there is provided a method of calibrating a growing item counting system on a moving vehicle comprising the steps of:

providing a vehicle having an apparatus configured to capture images of growing items and process the images to count the growing items on a given plant, plurality of plants or part of a plant;

comparing the counted items to an expected count; and,

if the counted number of items differs from the expected count by a first predetermined margin, prompting a user to perform a calibration process.

Advantageously, this ensures that the system is accurate in the assumptions that it makes. Preferably the expected count is a mean of previous validated trees/parts of trees, with the predetermined margin being a multiple of the standard deviation of that data set.

Preferably the calibration process is configured to receive a manual count and compare the manual count to the counted number of items. If the manual count differs by a second predetermined margin from the counted number of items, the system applies or updates a factor to the counted number of items to bring the counted number of items within the second predetermined margin. This may be an obscuration factor.

According to an eighth aspect there is provided a method of processing growing item image data comprising the steps of:

providing a camera on a moving vehicle, the camera configured to capture multiple sequential images of a growing item;

identifying a growing item in a first image;

processing the first image to obtain data relating to the growing item;

calculating a predicted motion vector of the first item relative to the frame of reference of the moving vehicle;

identifying the growing item in a second, subsequent image based on the predicted motion vector.

This allows duplicates to be discarded in e.g. the count.

the predicted motion vector may be based on the vehicle speed, or may be based on, or modified to account for, the path of the growing item from one image to a subsequent image.

### BRIEF DESCRIPTION OF THE DRAWINGS

An example apparatus and method according to the invention will now be described with reference to the accompanying figures in which:

FIG. 1 is a side view of a first vehicle mounted apparatus according to the invention;

FIG. 2 is a system schematic of the apparatus of FIG. 1;

FIG. 3 is a flow diagram of the method of operation of the apparatus of FIGS. 1 and 2;

FIG. 4 is a plan view of the apparatus of FIG. 1 in operation in an orchard;

FIG. 5 is an end view of part of the apparatus of FIG. 1 in operation in an orchard;

FIGS. 6a to 6d show steps in an image processing algorithm according to the invention;

FIGS. 7a to 7d show steps in an image processing algorithm according to the invention;

FIG. 8 is an end view of part of a second apparatus according to the invention;

FIG. 9 is an end view of part of a third apparatus according to the invention;

FIG. 10 is a plan view of part of a fourth apparatus according to the invention;

FIG. 11 is a plan view of part of a fifth apparatus according to the invention; and,

FIG. 12 is a plan view of part of a sixth apparatus according to the invention.

### DETAILED DESCRIPTION

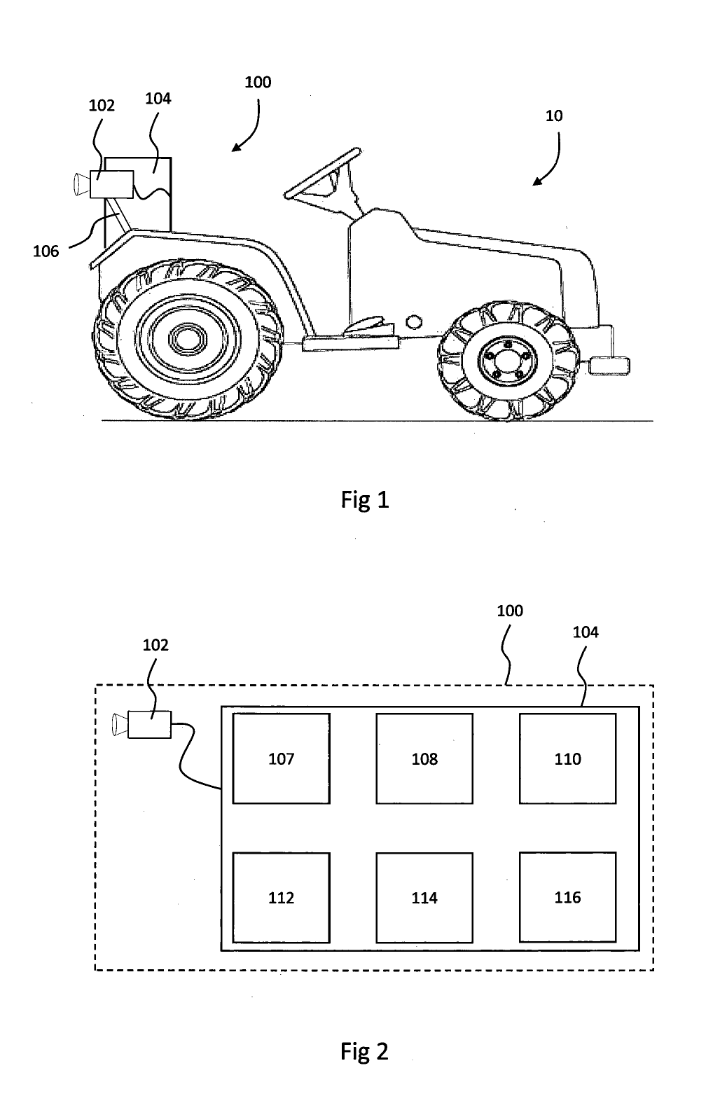

FIG. 1 shows a vehicle 10 having an apparatus 100 according to the present invention mounted thereon. The vehicle 10 is a compact off-highway vehicle, more particularly a quad which is well suited to travel on the bumpy and uneven ground surface found in an orchard.

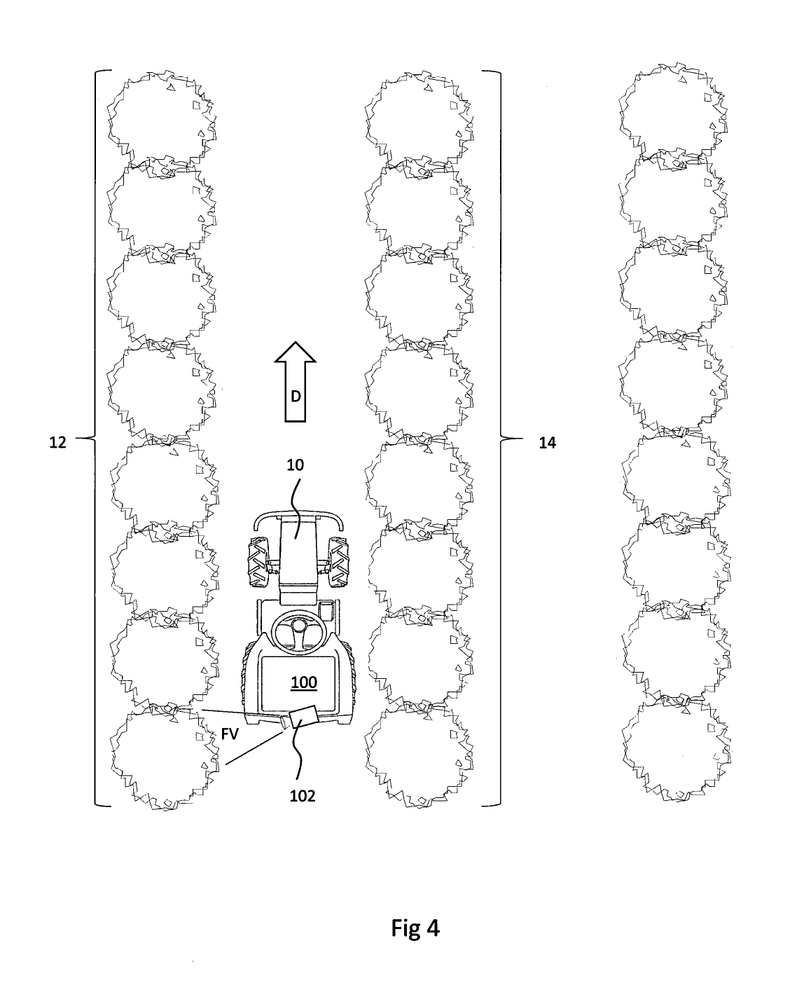

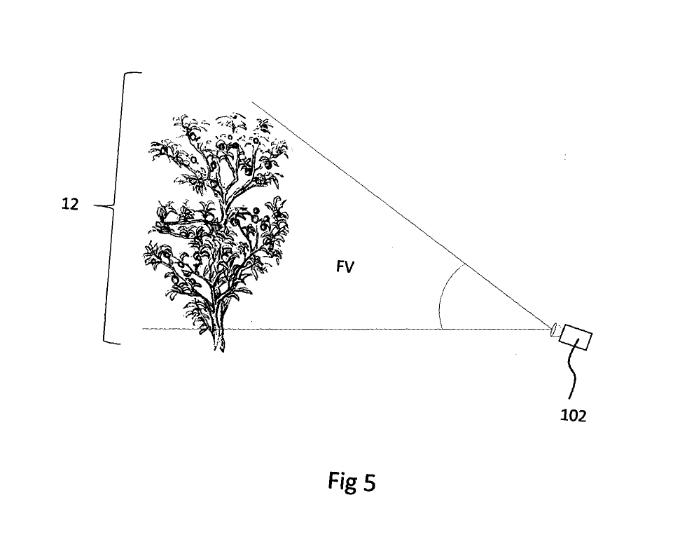

The apparatus 100 comprises a camera 102 and a computer 104. The camera 102 has a 2 megapixel resolution, and is capable of a frame rate of 20 Hz (most video cameras have a frame rate of at least 25 Hz). The camera 102 is mounted to the vehicle 10 on a shock/vibration absorbing mount 106. The camera is “ruggedized” by securing it in a protective enclosure, (such mounting systems and enclosures are known and will not be described in detail here). The mount 106 also includes the ability to manually adjust the position and direction of the camera 102. Referring to FIG. 4, the camera 102 is mounted such that when the vehicle 10 is driven in direction D between a first row of trees 12 and a second row of trees 14, the field of vision FV of the camera 102 is directed at the part of the tree containing the fruit, generally normal to the direction of travel D. FIG. 5 also shows that the camera is tilted towards the fruit growing part of the first row of trees 12.

The computer 104 is mounted to the vehicle 10. The computer 104 is a ruggedised Windows® PC, which is commercially available and is shown in more detail in FIG. 2.

Referring to FIG. 2, the computer 104 comprises a main CPU 107, a GPU 108, RAM 110, a solid state drive 112, a GPS chip 114 and a human machine interface (HMI) 116 comprising a graphical user interface (i.e. a display and input means). The SSD 112 stores the image processing software, which when executed on the CPU/GPU carries out the analysis as described below.

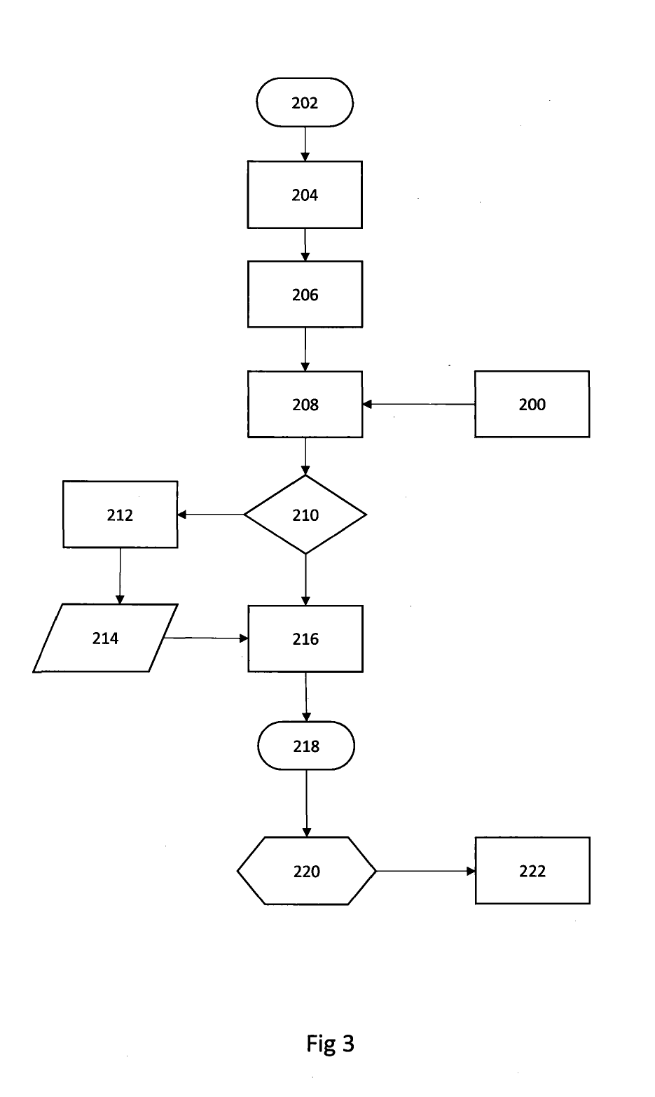

How these components are used is shown in FIG. 3. The software is loaded into the RAM 110. Before the analysis operation is started, at step 200 an operator uses the HMI 116 to set the parameters for the operation. These may include:
Apple count;
Apple type (i.e. breed), which assists the software in identifying the apples by colour and size (what “class 1” means varies by type);
Whether to analyse colour;
Whether to size the apples;
Mode—e.g. analyse for predicted harvest in which case identification and counting of “class 1” fruit is vital or “June drop” mode in which the number of immature fruit remaining on the tree is important.

This information toggles various settings in the software loaded into the RAM 110.

The system is started at step 202, and the vehicle 10 is driven in direction D at a speed of up to 10 kmph. At step 204, the camera 102 starts to capture 2 MP images at a frame rate of 20 Hz. It will be noted that the field of vision FV, the speed of the vehicle 10 and the frame rate are such that successive images overlap. In this way, fruit which may be obscured in one image may be visible in another (thus increasing the chances of detection). In this example, images are taken at about 14 cm intervals (20 Hz at 10 kmph).

At step 206 each image is stored in a ring buffer (or circular buffer) to buffer the image data stream. The ring buffer is provided to allow for variations in downstream processing time. The ring buffer acts as a “holding area” to ensure that no image is skipped or overwritten before it is analysed.

At step 208 the image processing software calls the next image in the buffer uses the parameters entered at step 200 to pre-process the images on CPU 107. Once an image has been called from the buffer it is not re-inserted (i.e. it is deleted from the buffer).

The first step of pre-processing is to create a co-occurrence matrix of the image. The co-occurrence matrix is based on the properties of hue and chroma (i.e. in the HC space, although red-green levels may be used as well to define a matrix in the RG space). The co-occurrence matrix represents a distribution of co-occurring values over the image.

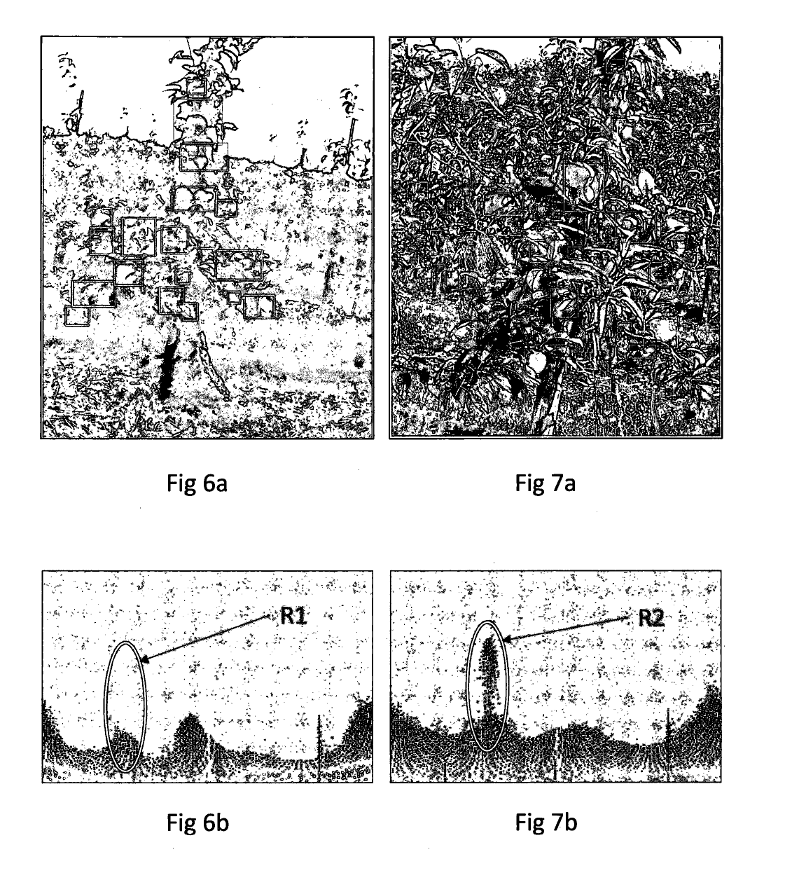

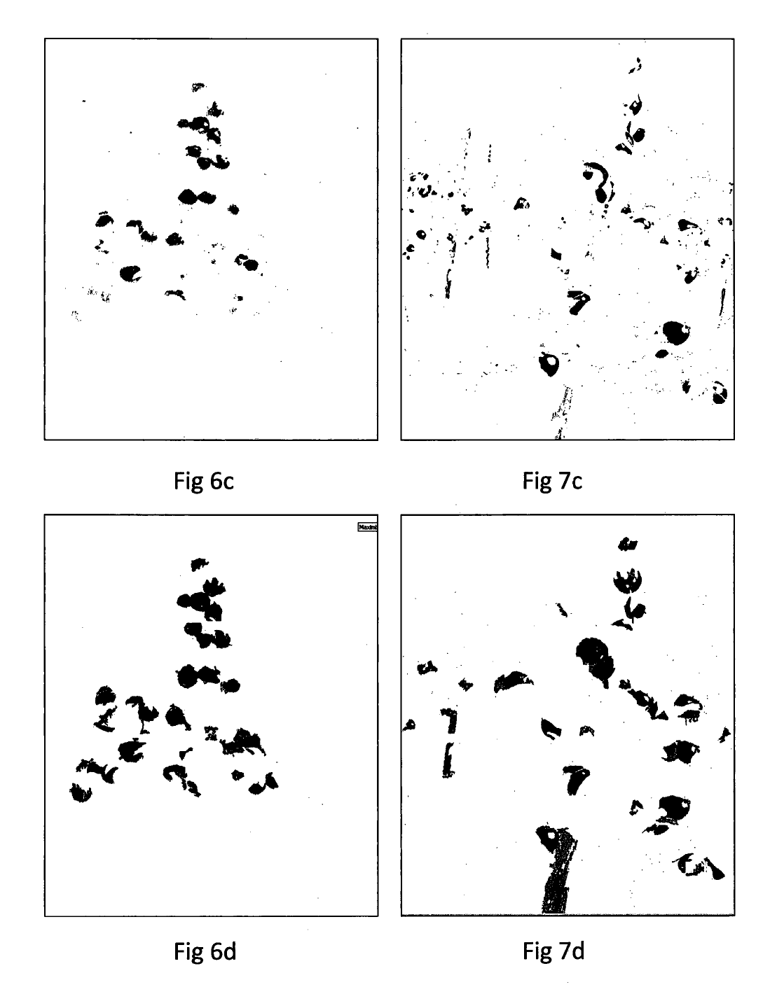

FIGS. 6a and 7a represent images of two different types of apple trees in different lighting conditions. The images are shown in greyscale (in reality these would be colour images). FIG. 6b shows a HC co-occurrence matrix for the image of FIG. 6a, and the peak at region R1 shows the region of the matrix which has the unique combination of properties signifying the presence of an apple in FIG. 6a. Similarly, region R2 of the matrix of FIG. 7b represents the apples of the image of FIG. 7a.

The step of co-occurrence analysis allows a series of “seed” pixels to be identified which have a high confidence of representing part of an apple. These are shown in FIGS. 6c (for the image of 6a) and 7c (for the image of 7a). The selection of these pixels will be dependent on a set of parameters which is selected by the user at step 200. If the user selects the breed of FIG. 6a, then a different selection criteria will be used to the breed of FIG. 7a because the apples have a different visual characteristic.

The next stage is segmentation—the aim of which is to identify pixels in the vicinity of the seed pixels which also represent parts of the growing item. The software uses two algorithms at this stage—the first is a basic flood fill segmentation algorithm. The flood fill algorithm seeks to find all similar pixels connected to seed pixels (or pixels already found by the algorithm), either directly or indirectly, using other less stringent criteria based upon hue and chroma. The flood fill algorithm is particularly useful if the image is noisy, or large differences in contrast. The second algorithm is a spiral algorithm whereby pixels are sequentially analysed in a spiral emanating from the seed pixel. The acceptance criteria are also less stringent values of hue and chroma. Using these two techniques, “blobs” of pixels which may be growing items are identified.

All these groups of pixels (“blobs”) are treated as separate objects and subjected to tests on size, aspect ratio and area etc in a refining step. For example, if a region of pixels is smaller than a given size, it may be filtered out—i.e. disregarded. Again, the parameters which are used for this refining step are dependent upon the user defined input at step 200. If the user wishes to use the system to identify immature apples during the “June drop”, then the minimum size is reduced. If the areas of pixels meet the criteria then they are labelled as possible apples. This results in the segmentation shown in FIGS. 6d and 7d. The rectangular boxes shown in FIGS. 6a and 7a are the apples identified after segmentation and refining as described above.

At step 210, specialist tasks are sent to the GPU for processing at step 212. The GPU carries out the following tasks to further refine the results. Software on the GPU carries out a characterisation step to examine shape, size, aspect ratio and colour. The software on the GPU uses more complex algorithms to disregard non-apples (i.e. non spherical objects). Solving of partial obscuration problems—e.g. sizing an apple if only half of it is visible by extrapolating its shape is also carried out by the GPU.

At step 214, the results are sent back to the main CPU 107 for final image processing at step 216 (where they are combined with the results of the image pre-processing step 208). The results represent a series of apple locations and characteristics derived from the image data.

At the final processing stage 216, apple association is carried out. Results from successive images are combined to eliminate duplicates.

It is clear that as the process is carried out, information is stripped from the image data leaving the apple data only. The images do not need to be stored, and are deliberately discarded to ensure that the limited space available on the computer 104 is used to best effect.

Data relating to all apples that have not been disregarded is combined with the location of the vehicle 10 as provided by the GPS chip 114 at step 218. At step 220 the data is added to a register stored on the memory 110.

The register is a database with entries corresponding to each fruit. The database fields may relate to size, colour etc. The register is converted to a visual map of fruit at step 222, which is displayed on the HMI 116 in real time. The driver of the vehicle is therefore provided with real time information regarding the counting process.

As well as this information, the HMI provides feedback on the status of the apparatus 100. If the vehicle is travelling too quickly, the computer 104 will not be able to process the images in time to generate the required accuracy. The main symptom of this is that the ring buffer will be constantly full, with previous images being overwritten instead of called by the image processing software. Therefore the HMI can provide a graphical depiction derived from the state of the ring buffer—e.g. with “green” being partially empty, “amber” being almost full and “red” being full—i.e. with images at risk of being overwritten before analysis.

The register can also be output as a CSV file for use with other farming software. The use of a GPU in conjunction with a CPU enables a very high processing speed. This system therefore analyses the images as they are recorded by the camera, i.e. in “real-time”.

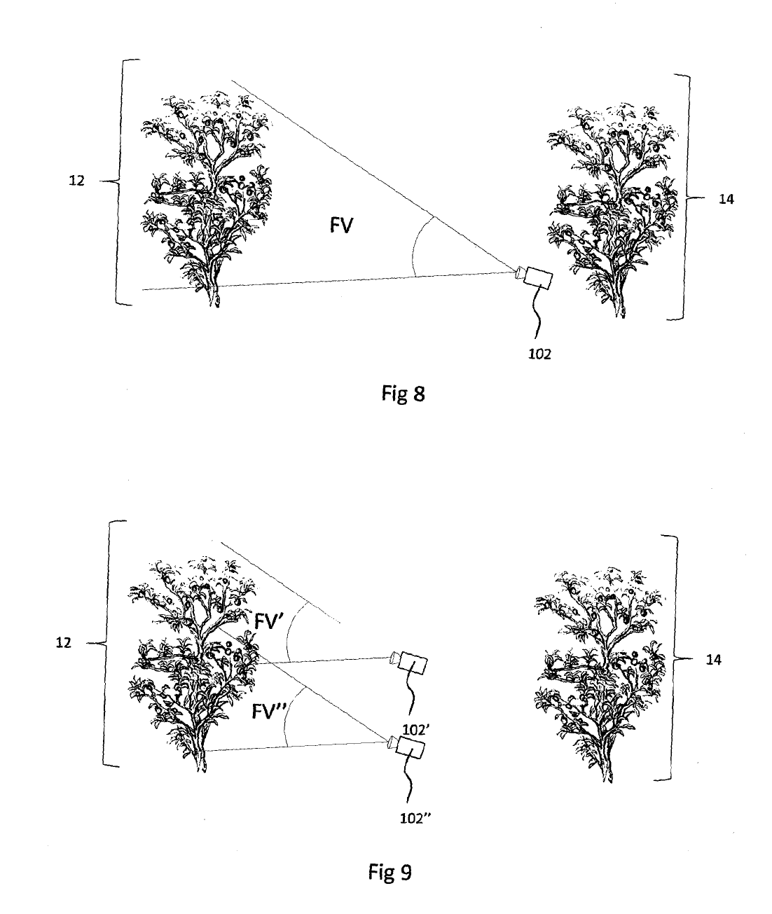

Various physical techniques may be employed to ensure that all the fruit are captured. Referring to FIG. 8, the camera 102 is positioned as far from the row of trees 12 as possible, and is in fact on the opposite side of the vehicle 10 (not shown) to the row 12.

In FIG. 9, two cameras 102′ and 102″ are shown placed above each other, whose fields of vision FV′ and FV″ can be combined to generate a full portrait image of the row of trees 12.

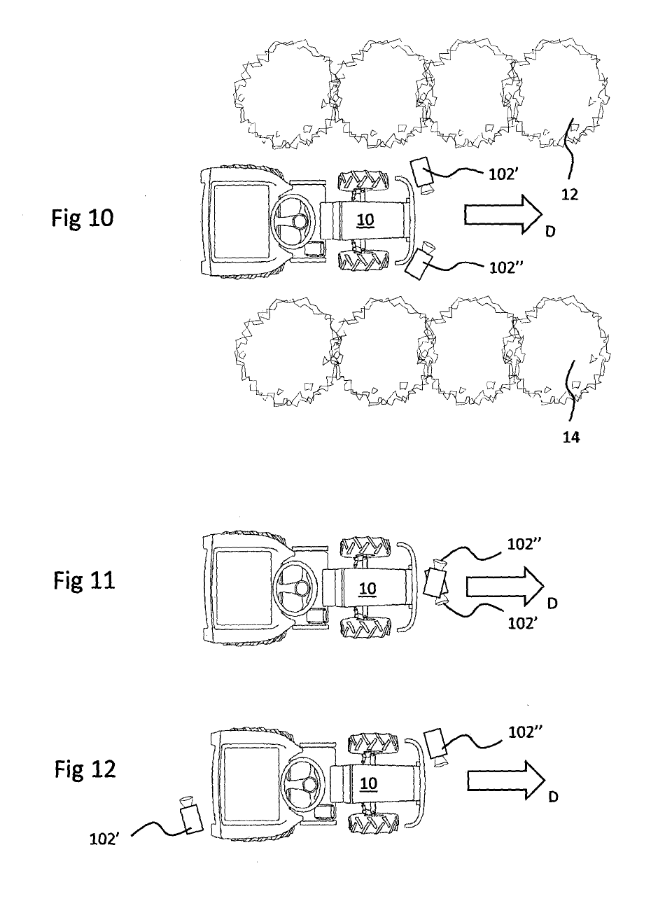

In FIG. 10 two cameras 102′ and 102″ are provided to look in opposite directions and thereby analyse both rows 12 and 14. The cameras are placed on the opposite side of the vehicle 10 to the row they are imaging for maximum field of vision.

In FIG. 11, like FIG. 10 a camera is provided for each row, but both are mounted at the centre of the vehicle 10.

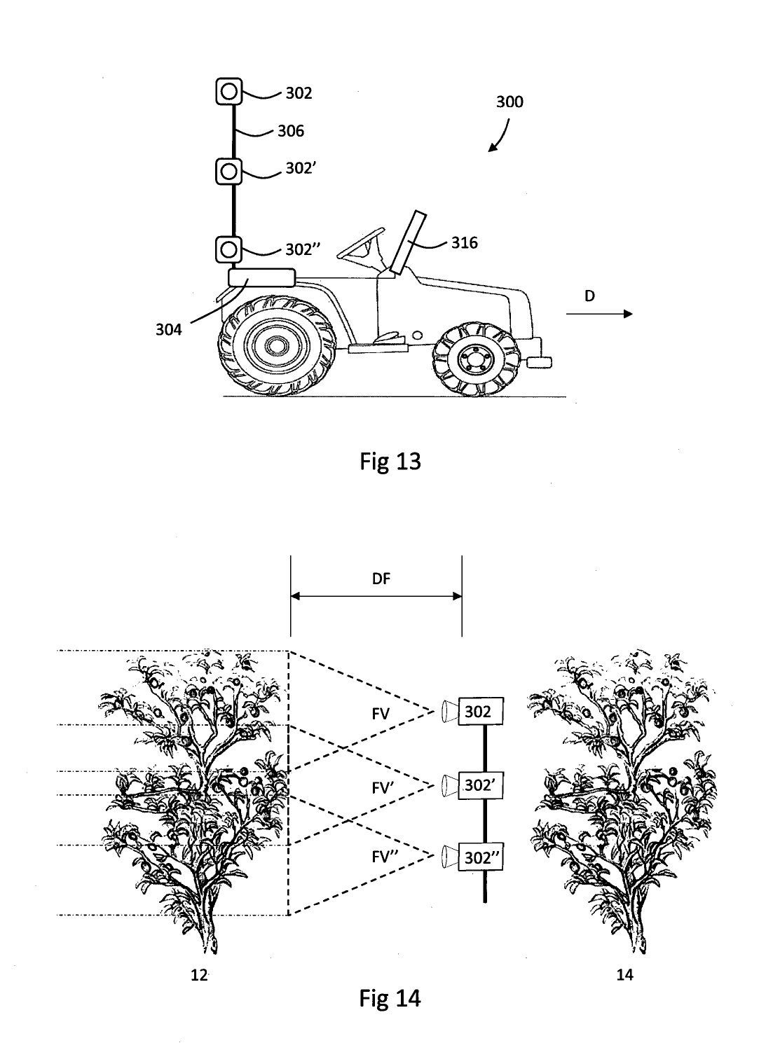

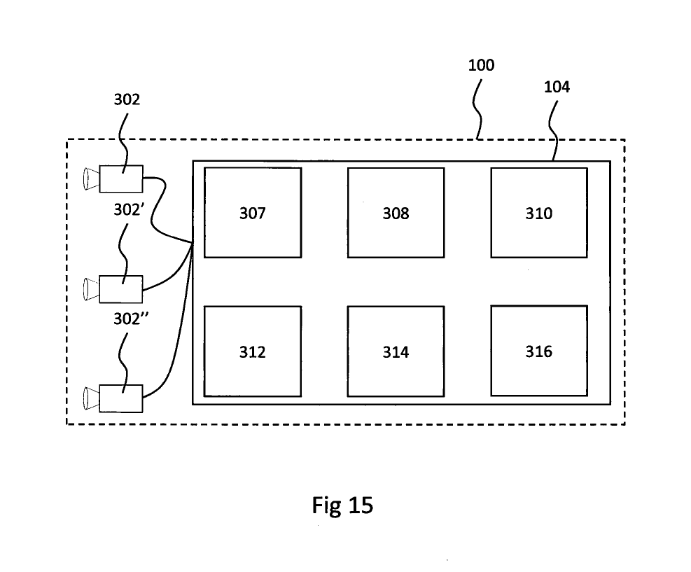

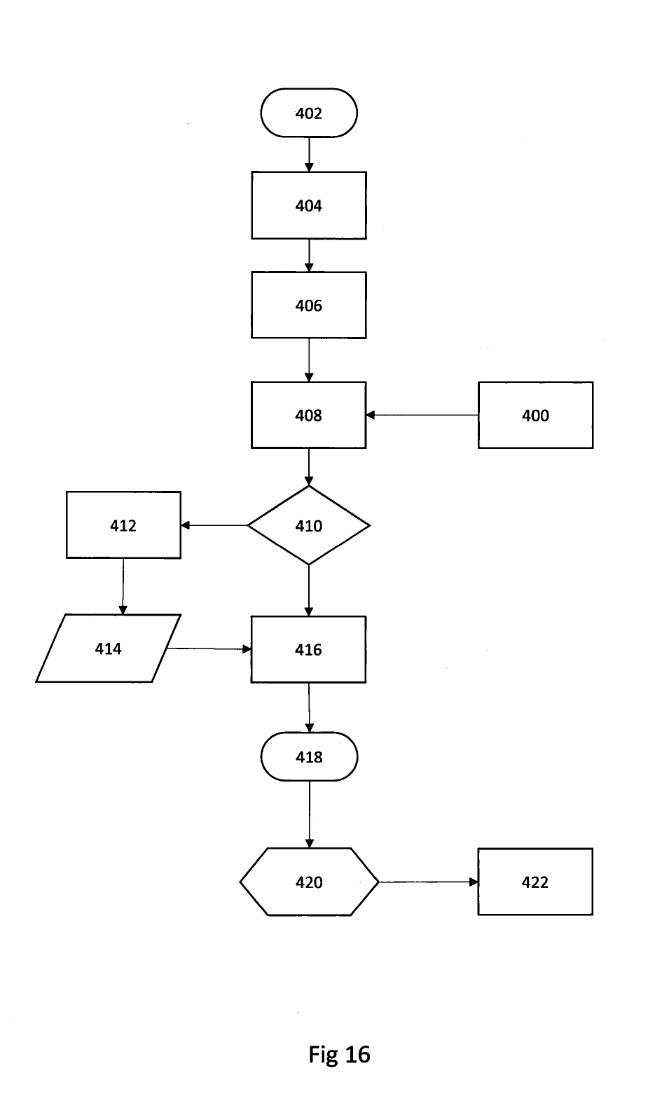

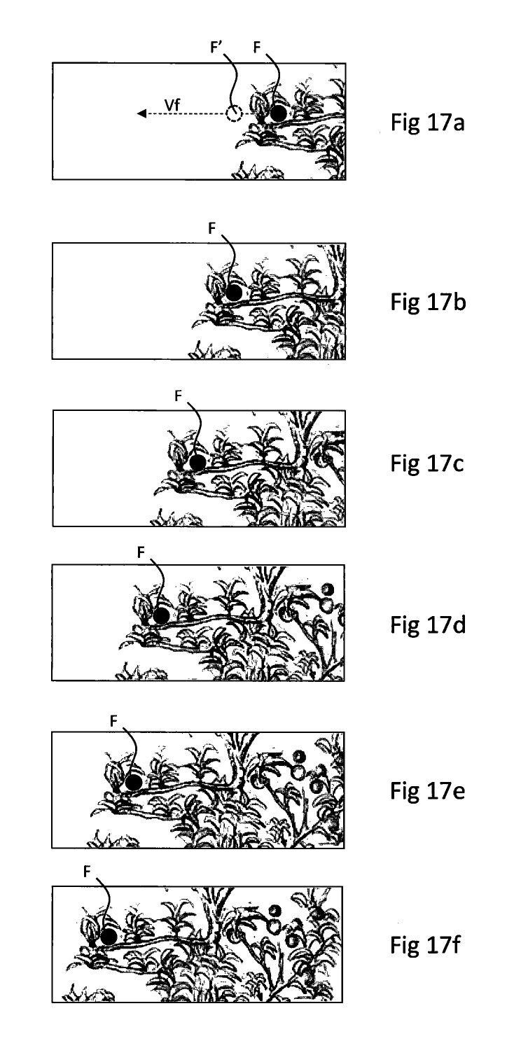

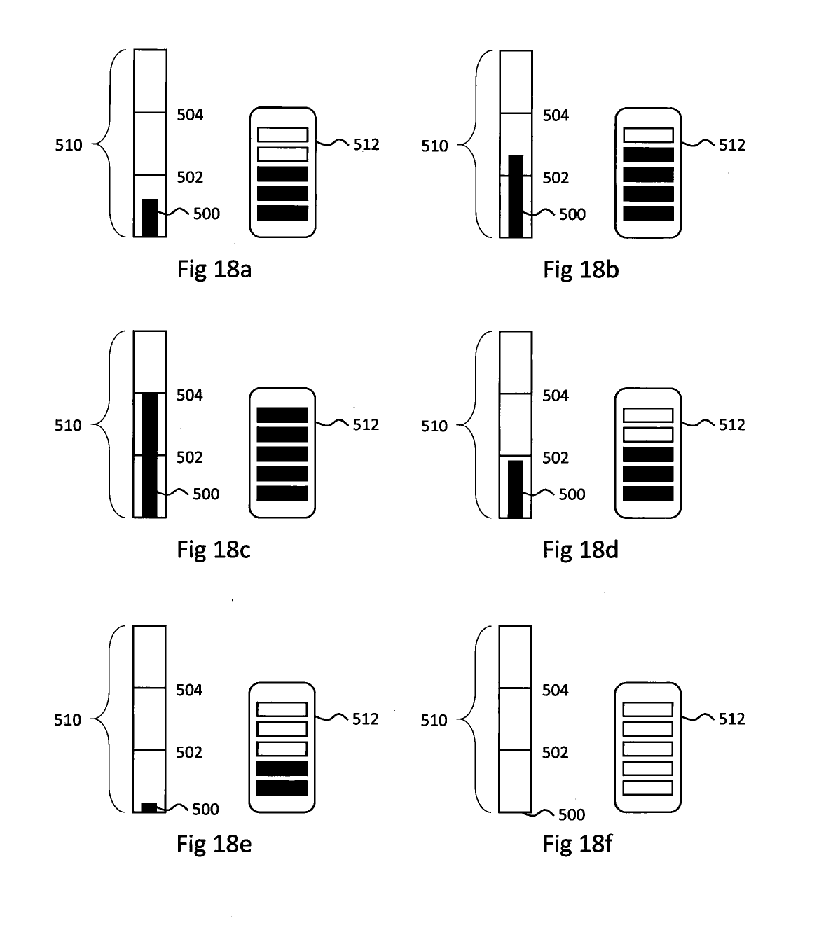

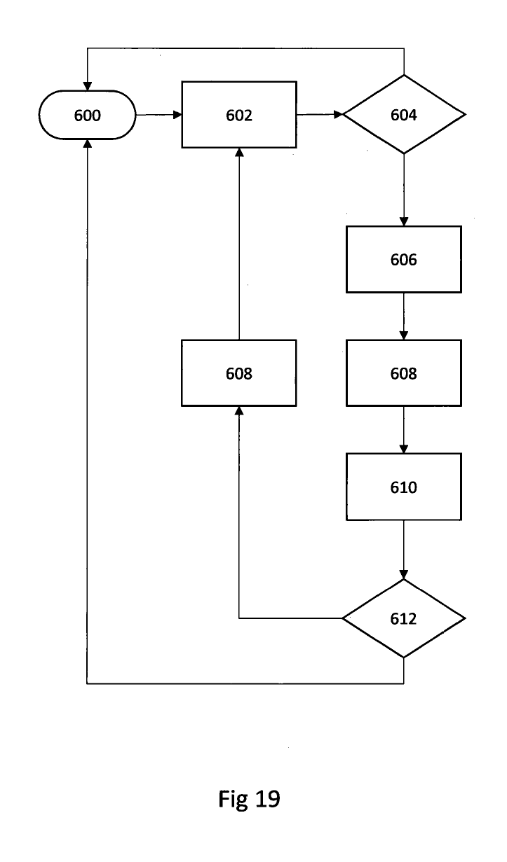

In FIG. 12, like FIG. 10 a camera is provided for each row, but both are mounted at opposite ends of the vehicle 10 in the direction of travel D.

Turning to FIG. 13, an example apparatus 300 according to the present invention is mounted on an off-road vehicle 10. The apparatus 300 is similar to the apparatus 100, and comprises first, second and third cameras 302, 302′, 302″ and a computer 304. The cameras 302, 302′, 302″ each have a 2 megapixel resolution.

The cameras 302, 302′, 302″ are mounted to the vehicle 10 on a camera mount pole 306 which in turn is mounted to the vehicle on a shock/vibration absorbing mount (not shown). The cameras are “ruggedized” by securing then each in a protective enclosure, (such mounting systems and enclosures are known and will not be described in detail here).

The cameras are mounted to the pole 306 such that they can be moved, although it will be understood that the cameras are spaced in a vertical line, and are all pointed in the same direction. Referring to FIG. 14, the cameras 302, 302′, 302″ are mounted such that when the vehicle 10 is driven in direction D between a first row of trees 12 and a second row of trees 14, the field of vision FV of the camera 102 is directed at the part of the first row of trees 12 containing the fruit. The cameras are usually pointed normal to the direction of travel D.

Each camera has a respective field of vision FV, FV′, FV″. The fields of vision FV, FV′ of the uppermost and middle cameras 302, 302′ partially overlap, as do the fields of vision FV, FV″ of the middle and lower cameras FV′, FV″.

The computer 304 is mounted to the vehicle 10. Like the computer 102, the computer 304 is a ruggedised Windows® PC, which is commercially available and is shown in more detail in FIG. 15.

Referring to FIG. 15, the computer 304 comprises a main CPU 307, a set of GPUs 308, RAM 310, a solid state drive 312, a GPS chip 314 and a human machine interface (HMI) 316 comprising a graphical user interface. The SSD 312 stores the image processing software, which when executed on the CPU/GPUs and carries out the analysis as described below.

How these components are used is shown in FIG. 16. The software is loaded into the RAM 310. Before the analysis operation is started, at step 400 an operator uses the HMI 316 to set the parameters for the operation. These may include (for apples):
Apple count;
Apple type (i.e. breed), which assists the software in identifying the apples by colour and size (what “class 1” means varies by type);
Whether to analyse colour;
Whether to size the apples;
Mode—e.g. analyse for predicted harvest in which case identification and counting of “class 1” fruit is vital or “June drop” mode in which the number of immature fruit remaining on the tree is important.

This information toggles various settings in the software loaded into the RAM 310.

The system is started at step 302, and the vehicle 10 is driven in direction D at a speed of e.g. up to 10 kmph. At step 404, the cameras 302, 302′, 302″ start to capture video at a frame rate of 20 Hz. It will be noted that the fields of vision FV, FV′, FV″, the speed of the vehicle 10 and the frame rate are such that successive images from each camera overlap. In this way, fruit which may be obscured in one image may be visible in another (thus increasing the chances of detection).

At step 406 each image is stored in an expandable buffer which is stored in the RAM 310. The buffer has a predetermined “standard” size to buffer the image data stream. If the buffer exceeds this size (e.g. if the speed at which the images can be processed falls behind the frame rate) the buffer is permitted to expand further into the RAM. At a certain predetermined maximum size, the buffer is not permitted to grow, and the oldest images are overwritten by new images. The buffer acts as a “holding area” to ensure that no image is skipped or overwritten before it is analysed, and may account for small variations in processing time.

At step 408 the image processing software calls the next image in the buffer and uses the parameters entered at step 400 to pre-process the images on CPU 307. Once an image has been called from the buffer it is not re-inserted (i.e. it is deleted from the buffer).

As with the previous embodiment, the first step of pre-processing is to create a co-occurrence matrix of the image. The co-occurrence matrix is based on the properties of hue and chroma (i.e. in the HC space, although red-green levels may be used as well to define a matrix in the RG space). The co-occurrence matrix represents a distribution of co-occurring values over the image.

At step 410, specialist image processing tasks are sent to the GPU for processing at step 412 as with the previous embodiment. In particular apple sizing is carried out at this step.

In order to relate the size of the apples on the image to the real-life size, the distance between the camera and apple is required. In this embodiment, the software is programmed to carry out a depth of field approximation (DF—the approximate distance to the trees—FIG. 14). This is calculated by a stereoscopic distance measuring using adjacent cameras—in this instance by images captured simultaneously by the cameras 302, 302′. Stereoscopic distance measuring is well known in the art, and will not be discussed further here. By looking at a detected object (e.g. an apple) in an overlapping field of vision (e.g. between FV and FV′), and with knowledge of the distance between the cameras 302, 302′, the distance DF can be approximated.

The distance FV is used when sizing all of the fruit in the images captured by the three cameras. In use, the distance between the closest and furthest apples is less than 0.5 m, and of using a single distance FV for all detected fruit across the three fields of vision FV, FV′, FV″ is sufficiently accurate.

At step 414, the results are sent back to the main CPU 307 for final image processing at step 416 (where they are combined with the results of the image pre-processing step 408). The results represent a series of apple locations and characteristics derived from the image data.

At the final processing stage 416, apple association is carried out. Results from successive images are compared to eliminate duplicates. This is performed by the CPU using the process shown in more detail in FIGS. 17a to 17f.

A series of frames are shown from the camera 302 moving from left to right in FIGS. 17a to 17f. As such, the image moves from right to left from this fixed point of view. If a fruit F is detected in FIG. 17a, the software is programmed to affix a velocity vector Vf to the fruit F. The vector Vf is usually opposite to the known velocity vector of the vehicle 10 (based on the measured vehicle speed). This provides an expected position of the fruit F: F′. If the fruit F appears at the expected position F′ (within a reasonable error) the system can discount it as a duplicate.

Once the fruit is detected in FIG. 17b at the expected position F′ (or thereabouts), the vector Vf can be modified based on the measured speed of the fruit. This can easily be calculated from the frame rate and distance traveled. This method of updating the fruit vector accounts for differences in object speed based on parallax. If the fruit is obscured in any given shot (e.g. by leaves) the last fruit vector Vf is used to predict its position and re-identify it once it reappears.

It is clear that as the process is carried out, information is stripped from the image data leaving the apple data only. The images do not need to be stored, and are deliberately discarded to ensure that the limited space available on the computer 104 is used to best effect.

Data relating to all apples that have not been disregarded is combined with the location of the vehicle 10 as provided by the GPS chip 114 at step 218. At step 220 the data is added to a register stored on the memory 110.

The register is a database with entries corresponding to each fruit. The database fields may relate to size, colour etc. The register is converted to a visual map of fruit at step 222, which is displayed on the HMI 116 in real time. The driver of the vehicle is therefore provided with real time information regarding the counting process.

The apparatus 300 further comprises a speed governing system 500 as shown in FIGS. 18a to 18f. This system is carried out on the software of the apparatus alongside the other functions described above. As discussed, t is desirable for the vehicle 10 to travel as quickly as possible for the most efficient operation.

As mentioned above, the apparatus has an image data buffer 500 which has a standard size allocation 502 in the RAM 510, and an expanded size 504. The buffer is not permitted to expand beyond the expanded size 504. On the right hand side of FIG. 18a is a speed status display 512 which is provided on the HMI 316.

In FIG. 18a, the buffer size is within the standard allocation 502. As such, the speed status display shows that the speed is correct—i.e. it shows three out of five bars and is coloured green.

In FIG. 18b, although the speed of the vehicle has not changed, the number of fruit per image has increased, thus increasing the processing burden on the processors. This decreases the turnaround time per frame, and as such the buffer starts to fill quicker than the images are being processed. As the buffer 500 grows into the “expanded buffer” region 504, the status changes to amber, and four bars are filled. This is a warning to the driver that the vehicle speed is too high, and to slow the vehicle.

In FIG. 18c, the buffer has filled the standard and expanded allocation on the RAM. Images are being overwritten in the buffer and the status 512 shows red to alert the driver to slow down immediately. An audible warning is also provided.

In FIG. 18d, the vehicle has slowed and the buffer size has decreased to normal. The status 512 is normal.

In FIG. 18e, the processors have caught up to the extent that the buffer size is well below (10% of) the standard allocation. The status display 512 drops to two bars, and turns amber, encouraging the driver to speed up to make the process more efficient (i.e. the apparatus 100 can handle a higher speed).

In FIG. 18f, the buffer 500 has almost emptied, and the display turns red, showing no bars and encouraging the driver to speed up significantly.

In this way, because the system demand is dependent on the amount of fruit data in the images, the vehicle can be sped up and slowed down depending on processor demand to provide the most efficient process.

The apparatus 300 utilises several assumptions to provide a fruit count. Firstly, only one side of a row of trees may be scanned. The number of fruit counted by the system is multiplied by an obscuration factor to estimate the total count of growing items. This accounts for apples on both sides of the tree which are obscured by leaves etc. and are simply not visible from the vehicle 10.

The obscuration factor varied depending on the type of fruit, trees and environmental conditions (time of year etc.). Therefore, although an assumption can be made, calibration is highly beneficial.

As such, the software of the apparatus 300 has a calibration function which operates with reference to FIG. 14.

At step 600 the apparatus 300 counts the apples visible on a given tree (over several frames). The counted number of apples is multiplied by an obscuration factor at step 602 to provide an adjusted estimated count. At step 604 a check is carried out to determine whether the estimated count for that tree is within an error margin of an average count. If the estimated count is within the error margin, then the process is repeated (top line from 604 to 600). If the estimated count is outside the error margin, then the calibration process is started.

The average count and error margin will start as user-defined values (possibly based on previously collected and verified data for that cultivar), but as more data is amassed this will be updated to be the mean μ of the calibrated (i.e. manually counted trees) in that project with a standard deviation σ. The error margin is set as a factor of the standard deviation σ, in this case ±2σ.

At step 606 a message is displayed on the HMI to the driver to stop and exit the vehicle. He will then manually count the apples on the tree just scanned at step 608. At step 610 the user enters the number into the HMI. At step 612, the software compares the manual count to the estimated count to determine whether the estimated count was correct. If the estimated count was within an acceptable error of the manual count, the scanning resumes as before. If not, then the obscuration factor is adjusted at step 608 to bring the estimated count in line with the manual count and the process resumed.

Variations fall within the scope of the present invention.

It will be understood that when a single program, memory or processor is described, the functionality of those elements of the invention may be distributed across more than one such element. For example, image analysis may be carried out by two different programs sequentially or in parallel. Similarly, the system architecture may benefit from parallel processing, in which case more than one processor will be provided.

The term “software” is used in a broad sense, and the described functionality may be carried out by e.g. firmware.

The apparatus may be mounted to a different type of manned vehicle, for example a tractor. The vehicle may be on rails or tracks instead of wheels. Alternatively the apparatus may be mounted to an unmanned guided vehicle. The benefit of using an automatically guided vehicle is that the same path will be followed for repeat analyses (e.g. the next season). This is beneficial as it provides more comparable results. The apparatus may be mounted to an aircraft or UAV.

The computer 104 may have wireless capability (Wi-Fi or Bluetooth®) in order to provide an upload capability to a stationary or “base” computer for subsequent analysis and storage.

Instead of a visual speed display as shown in FIGS. 18a to 18f, a direct command may be provided to an automated speed control. As such the speed of the vehicle may be automatically varied based on the processor demand (indicated by buffer size).

What is claimed is:

1. An apparatus for analysis of growing items, the apparatus suitable for installation on a vehicle and comprising:
at least one camera configured to capture multiple sequential images of growing items;
a memory comprising an image buffer for storing the images;
a processor;
software, which when executed on the processor is configured to repeatedly:
call an image from the image buffer;
process the image to identify individual growing items to create item data relating to individual growing items;
update a growing item database by adding the item data to the growing item database;
wherein during vehicle motion:
the software is configured to run whilst the camera captures further images; and,
at least some of the images are discarded once they have been processed by the software.

2. An apparatus according to claim 1, comprising a GPS receiver for generating location data, in which the software is configured to combine the item data with location data and update the growing item database with the combined item data and location data.

3. (canceled)

4. An apparatus according to claim 3, in which the software is configured to identify and disregard growing items which were previously added to the growing item database by carrying out the following steps:
identifying a growing item in a first image;
predicting the position of the growing item in a second, subsequent image; and,
disregarding the growing item in the subsequent image if the position matches the predicted position within a predetermined margin of error.

5. An apparatus according to claim 4, in which the step of predicting the position of the growing item is carried out using a velocity vector based on the speed and direction of a vehicle on which the apparatus is mounted.

6. An apparatus according to claim 5, in which the velocity vector is refined by calculating the speed and direction of the growing object based on changes in its position between at least two images.

7. An apparatus according to claim 1, in which images are removed from the buffer when they are called by the software.

8.-13. (canceled)

14. An apparatus according to claim 1, in which the software is configured to identify seed pixels in the image using a co-occurrence matrix, and to carry out a segmentation step to identify further candidate pixels proximate the seed pixels.

15.-17. (canceled)

18. An apparatus according to claim 14, in which in the segmentation step, an algorithm is selected from the list comprising a flood fill algorithm and a spiral algorithm based on a set of segmentation parameters less stringent than the seed parameters.

19. (canceled)

20. An apparatus according to claim 18, in which the software is configured to filter individual regions of pixels are based on size, and regions below a certain size are disregarded.

21. An apparatus according to claim 20, in which the software is configured to produce item data by analysis of at least one of the size, shape, colour and aspect ratio of the regions of pixels.

22. An apparatus according to claim 21, in which the software is configured to identify partially obscured growing items and to extrapolate at least one of their size and shape.

23.-27. (canceled)

28. An apparatus according to claim 1, in which the at least one camera comprises a first camera and a second camera having an overlapping field of vision with the first camera, in which the software is configured to calculate a depth of field between the cameras and the growing items using a stereoscopic measuring technique based on an overlap between simultaneously captured images.

29. An apparatus according to claim 28, in which the software is configured to process the image to size individual growing items based on the calculated depth of field.

30. An apparatus according to claim 29, in which individual growing items outside the overlapping field of vision are sized based on the calculated depth of field.

31. An apparatus according to claim 1, configured to:
determine a status of the buffer;
output a speed feedback signal based on the status of the buffer.

32. An apparatus according to claim 31, in which in determining the status of the buffer the apparatus determines the amount of data in the buffer.

33. An apparatus according to claim 31, comprising a user display, in which the feedback signal is fed to the user display.

34.-35. (canceled)

36. An apparatus according to claim 1, configured to:
count the number of growing items on a given plant, plurality of plants or part of a plant;
compare the counted items to an expected count; and,
if the counted number of items differs from the expected count by a first predetermined margin, prompt a user to perform a calibration process.

37. (canceled)

38. An apparatus according to claim 36, in which the calibration process is configured to receive a manual count and compare the manual count to the counted number of items, in which if the manual count differs by a second predetermined margin from the counted number of items, to apply or update a factor to the counted number of items to bring the counted number of items within the second predetermined margin.

39.-41. (canceled)

42. A method of analysis of growing items comprising the steps of:
providing a vehicle;
providing an apparatus mounted on the vehicle, the apparatus comprising:
at least one camera configured to capture multiple sequential images of growing items;
a memory comprising an image buffer for storing the images;
a processor;
software, which when executed on the processor is configured to repeatedly:
call an image from the image buffer;
process the image to identify individual growing items to create item data relating to individual growing items; and
update a growing item database by adding the item data;
causing the vehicle to move, and whilst the vehicle is in motion;
running the software whilst the at least one camera captures images; and
discarding at least some of the images once processed by the software.

43.-109. (canceled)
